(19) United States
(12) Patent Application Publication
(10) Pub. No.: US 2014/0017341 A1
(43) Pub. Date: Jan. 16, 2014

(54) COMPOSITION AND METHOD FOR TREATING FAT TISSUES AND INFLAMMATORY PROCESSES

(71) Applicant: Brigitte Gourlaouen, Paris (FR)

(72) Inventor: Brigitte Gourlaouen, Paris (FR)

(21) Appl. No.: 13/880,663

(22) PCT Filed: Oct. 19, 2011
(86) PCT No.: PCT/FR2011/052446

§ 371 Date: Sep. 20, 2013

(30) Foreign Application Priority Data
Oct. 19, 2010 (FR) 1058532

(51) Int. Cl. A61K 36/9066; A61K 36/888; A61K 36/73; A61K 31/522; A61K 36/77; A61K 36/81; A61K 36/23; A61K 36/82; A61K 36/07; A61K 36/47; A61K 31/353; A61K 31/4525; A61K 36/704; A61K 36/708; A61K 36/484; A61K 36/60; A61K 36/03; A61K 36/41; A61K 36/074; A23L 1/30; A23L 2/52; A61K 36/88

(52) U.S. Cl. CPC A61K 36/9066; A61K 36/88; A61K 36/888; A61K 36/73; A61K 31/522; A61K 36/77; A61K 36/81; A61K 36/23; A61K 36/82; A61K 36/07; A61K 36/47; A61K 31/353; A61K 31/4525; A61K 36/704; A61K 36/708; A61K 36/484; A61K 36/60; A61K 36/03; A61K 36/41; A61K 36/074; A23L 1/30; A23L 2/52
USPC 424/702; 424/195.15; 424/729

(57) ABSTRACT

The process of the invention relates to a kit of two cosmetic, pharmaceutical, veterinary and food compositions, designed for slimming or for preventing and/or repairing inflammatory mechanisms, one of the compositions comprising at least one sirtuin activator and at least one HSP activator, and the other composition comprising at least one sirtuin inhibitor and at least one HSP inhibitor.

The said compositions are intended to be delivered in a chronomodulated pattern. They are particularly effective in combination with one another for controlling fat tissues, adipose, fibrous or aqueous cellulite, cell aging and inflammatory processes.

The invention relates to a product for cosmetic, pharmaceutical, food or veterinary use, intended for loss of the fatty volume contained in the tissues and/or intended for reducing inflammatory processes. This product comprises at least two compositions, one being administered during the day, preferably in the morning, and the other at night, preferably in the evening. This product makes it possible to improve cell function by virtue of a double mode of action: adaptive resynchronization of the cell, and direct or indirect lipolysis mechanisms.

The invention also relates to a cosmetic care process or a therapeutic treatment method using these compositions.

The product combines two compositions: sirtuin inhibitors and HSP (heat shock protein) inhibitors are administered during the day, while sirtuin activators and HSP activators are provided during the nocturnal period. The cutaneous application or the oral intake of the compositions is carried out according to a chrono-modulated scale over twenty-four hours, according to the individual chronobiological rhythm of the human being or of the animal, in order to ensure the optimum effect of the active ingredients.

### STATE OF THE ART

Various types of compositions that are of use in the field of slimming and of increasing the lifetime of cells are known and used on daily basis. However, it is still relatively uncommon to use, in combination with one another, compositions of which the biological effects are complementary.

A method for cosmetic body treatment to enhance the silhouette and to develop the female bust using two cosmetic compositions, one having lipolytic properties and the other being capable of retaining fats in the adipocytes has been described in international application WO 2004/037221. The purpose of this treatment is to increase the volume of zones judged to be too small and to decrease the volume of a zone judged to be too large. The treatment causes, for example, lipolysis of the fats at the level of the hips, and capture of the fats thus released in order to fix them at the level of the breasts. Caffeine, tocopheryl nicotinate, kola extract, carnitine, vitamin E and ginkgo biloba are used as lipolytic agent.

The action of sirtuins has not really been studied and the preparation of effective compositions comprising sirtuin modulators constitutes a technical problem in its own right that must be solved.

To date, compositions which have a sirtuin-inhibiting or sirtuin-activating activity have already been described, for example in application US 2005/0136537.

In application FR 2 906 143-A1, a cosmetic treatment process has been proposed which consists in applying, during the day, a composition containing sirtuin inhibitors and, at night, a composition containing sirtuin activators. According to the teaching of that document, the process makes it possible to resynchronize the circadian rhythms of the skin and to optimize its activity. That document does not teach that such compositions are effective for treating cellulite or for preventing an inflammatory process.

However, the uses of sirtuin modulators described to date in the prior art do not take into account a certain number of factors, including in particular: the memorization of repeated stresses, the potential differentiation of certain cells (fibroblasts, mesenchymal, monocytes) into adipocytes, and the inflammatory component of the cell tissue (peroxisome proliferator-activated) which, in the long term, can cause the appearance of inflammatory disease (atherosclerosis, type III diabetes) and reduce the effectiveness of the activity sought. It is in particular necessary to modulate the negative effects of the hyperactivity caused by sirtuins, which cause an exaggerated stimulation of the apoptotic P53 genes and the acceleration of metabolic senescence.

### PURPOSES OF THE INVENTION

Thus, the inventor has desired to solve the novel technical problem consisting in improving and/or controlling the action of compositions comprising slimming agents and/or agents for preventing inflammatory phenomena, for the purpose of preserving cell integrity, in particular by having combined and alternate actions on the activity of cells, and in particular adipocytes.

The aim is to solve this problem in particular when these compositions comprise sirtuin modulators.

The purpose of the invention is to solve the novel technical problem consisting in improving the slimming activity of compositions containing sirtuin inhibitors.

The purpose of the invention is also to provide a cosmetic, pharmaceutical, food or veterinary composition which has inflammatory-process-preventing properties, in particular a repairing or protective effect on the integrity of the cell.

In particular, the purpose of the invention is to preserve and improve the beneficial activity of sirtuin modulators while seeking to limit their harmful effects as much as possible.

According to a first embodiment, the purpose of the invention is to seek to control the activity and/or reduce the negative effects of a composition comprising sirtuin activators.

According to the second embodiment, the purpose of the invention is to seek to control the activity and/or reduce the negative effects of a composition comprising sirtuin inhibitors.

The purpose of the invention is to solve this technical problem in a simple, inexpensive and industrially reproducible manner, while advantageously taking into account Community directive 76/768/EEC relating to cosmetic products.

### DESCRIPTION OF THE INVENTION

The inventor has observed, unexpectedly, that the beneficial effects of sirtuins, such as the slimming effect and/or the effect of preventing inflammatory processes, can be improved in accordance with the biological rhythm of the cell, by combining with them a heat shock protein (HSP) modulator.

Thus, according to the invention, at least one HSP inhibitor and at least one sirtuin inhibitor are combined with at least one HSP activator and at least sirtuin activator, in particular in accordance with the alternation of the nycthemeral cycle.

Thus, the process of the present invention involves a kit of slimming and/or inflammatory-process-preventing compositions, which are in particular cosmetic, pharmaceutical, food or veterinary compositions, comprising:
a diurnal composition containing at least one inhibitor of at least one type of sirtuin (termed “sirtuin inhibitor”) and at least one inhibitor of at least one type of HSP (termed “HSP inhibitor”), and
a nocturnal composition comprising at least one activator of at least one type of sirtuin (termed “sirtuin activator”) and at least one activator of at least one type of HSP (termed “HSP activator”).

The present invention also covers these compositions separately, i.e., on the one hand, a slimming and/or inflammatory-process-preventing composition, comprising at least one sirtuin inhibitor and at least one HSP inhibitor, advantageously intended to be applied during the day, and, on the other hand, a slimming and/or inflammatory-process-preventing composition comprising at least one sirtuin activator and at least one HSP activator, advantageously intended to be applied at night.

According to a second aspect, the present invention also covers abovementioned compositions, advantageously slimming and/or inflammatory-process-preventing compositions, also comprising at least one slimming agent, in particular a lipolysis-activating agent.

According to a third aspect, the present invention also relates to the abovementioned compositions, advantageously cosmetic slimming and/or inflammatory-process-preventing compositions, also comprising at least one vascular activating agent.

According to a fourth aspect, the invention also covers the use of a combination of at least one sirtuin modulator and of at least one HSP modulator as active ingredients, in a slimming composition or a composition for prevention of an inflammatory process, comprising at least one slimming agent and at least one vascular activating agent.

According to one embodiment, at least one HSP inhibitor and at least one sirtuin inhibitor are used as active ingredients of a slimming care composition or inflammatory-process-preventing composition, advantageously intended to be applied during the day.

According to one embodiment, at least one HSP activator and at least one sirtuin activator are used as active ingredients of a slimming care composition or inflammatory-process-preventing composition, advantageously intended to be applied at night.

### DEFINITIONS

The terms “sirtuin inhibitor/activator” are intended to mean a compound which has properties of inhibition/activation (respectively) of at least one protein of the sirtuin family, or a compound which mimics the inhibition/activation (respectively) of at least one protein of the sirtuin family, in particular sirtuin 1 and sirtuin 2, but also sirtuins 3 to 7. For the purposes of the invention, a sirtuin modulator can also be an agonist (synonym of activator) or an antagonist (synonym of inhibitor) of STACs (cell survival triggering compounds). STACs (sirtuin-activating compounds) are enzymes which use NAD+ to deacetylate proteins. Sirtuins 2 will act, via an allosteric mechanism, with STACs and associate with one another to increase the cell defenses against stress. STACs can be triggered via a stress mechanism, in a manner totally independent of that of sirtuins.

The terms “HSP inhibitor/activator” are intended to mean a compound which has properties of inhibition/activation (respectively) of at least one protein of the HSP family, or a compound which mimics the inhibition/activation (respectively) of at least one protein of the HSP family. The HSP inhibitor/activator is preferably an inhibitor/activator of one of the following HSPs: HSP70, HSP90, HSP100 and HSPsmall.

The term “protective on cell integrity” is intended to mean all the mechanisms of prevention against inflammatory processes.

The term “vascular activating agent” is intended to mean an agent which promotes blood circulation, which is favorable to vascular vasodilatation, or favorable to an increase in vascular flow rate in the tissues concerned or which mimics the mechanisms of a sporting activity.

The terms “inhibitor/activator” are intended to mean inhibiting, respectively activating, a protein under consideration, in particular via inhibition/activation of the expression of the gene of this protein, inhibition/activation of the translation of the mRNAs of this gene, and/or inhibition/activation of the activity of the protein.

The term “diurnal composition” is intended to mean a composition intended to be applied during the day, preferably in the morning between 6 am-11 am.

The term “nocturnal composition” is intended to mean a composition intended to be applied at night, preferably in the evening between 6 pm-midnight.

The combination of a sirtuin activator with an activator of an HSP protein according to the invention advantageously makes it possible to obtain the following effects. Stimulating sirtuins creates a chemical stress which abolishes the protective action of the P53 gene. The stimulation of the constitutive heat shock proteins (HSPs) which is proposed in the context of the invention therefore makes it possible to control the risks of things getting out of hand, associated with the hyperactivity of sirtuins that can, depending on the situation, promote or prevent the appearance of harmful phenomena (cancer). The activation of inducible HSPs makes it possible memorize stresses and to control them better.

Inhibiting sirtuins could trigger an increased activity of the apoptotic (programmed cell death) P53 genes. The concomitant inhibition of HSPs makes it possible to limit cell autophagy mechanisms from running out of control, and the consequences thereof on replicative aging and on metabolic senescence.

According to the process of the invention, it is recommended to modulate the activity of sirtuins with HSP modulators, taking into account the biological clock of the cell.

It is assumed that the sirtuin inhibitors will, via the P53 genomic pathway, activate HSL (Hormone-Sensitive Lipase)-dependent lipolysis and slow down cell activity while increasing the preservation of the energy capacities of the cell. The P53 gene is a general controller of cell death, but the protection that it exerts accelerates aging moderately and does not interfere with the lifetime of cells.

The invention is intended to cover the use of at least one sirtuin inhibitor as a slimming active ingredient in combination with an HSP inhibitor, especially in the compositions of the present invention in particular intended to be administered in the morning.

The use of the HSP inhibitors also makes it possible to facilitate adipocyte emptying by decreasing FA (fatty acid) storage, and to eliminate the risk of repeated cell stress memorization.

The enzymatic activators of sirtuins (Sir2 deacetylases) will mimic calorie control, enabling, in the nocturnal phase, a decrease in the fat store contained in the adipocytes, and blocking of the maturation of pre-adipocytes, in particular when they are combined with at least one HSP activator. It is known that the action of the Sirt 1 and Sirt 3 genes have an action on calorie control (inhibition of adipogenesis and activation of adipocyte lipolysis), and, by virtue of the inhibition of uncoupling protein 2 (UCP2), the Sifts 1 upregulate insulin secretion. The inflammatory processes, by virtue of the activation of peroxisome proliferator-activated receptors alpha (PPARs alpha) and of the transcription factors Forkhead box O (FOXO) 1, 3, 4, will decrease.

With regard to slimming, a slim woman exhibits areas of cellulite which sometimes stand out more than in a woman who is overweight. Thus, it is essential to distinguish a weight loss (by calorie control or by simple loss of water) from a slimming down (loss of fat volume). Their mechanisms are complementary and their effects join together in the prevention of inflammatory processes.

The inventor has observed that most slimming preparations act especially on the venous side and little on the micro-arterial side. They act on veno-lympathic edematous statis (excess water), but little on blood flow rate, and therefore not on alpha-2 receptors, nor on NEFA (nonesterified FA) exit. As it happens, lipomobilization depends essentially on arterial blood flow rate. It has been observed that the combination with a vascular activating agent makes it possible to significantly improve the slimming properties of a composition according to the invention. By virtue of an increase in the blood flow rate of a tissue, an increase in body temperature, blocking of alpha-2 receptors, increased release of NEFAs (hydrophilic triacylglycerols) responsible for insulin resistance of tissues, reduced risks of glycation responsible for excessive proliferation of collagen fibers and the like, and increased HSPs are observed. Tissue lipolysis (blocking of alpha-2 receptors and reduction of adipogenesis) is reinforced by adapting the galenic of the composition, and by combining therewith a vascular activating agent. The contact time of the circulating sugars with the adipocyte membranes is important in the glycation process and appears to be largely responsible for glycation, without suffering from diabetes.

The increase in vascular flow rate will trigger the activation of HSPs and that of PPARs, thus blocking the proliferation of smooth muscular cells, by curbing the activity of the telomerase enzyme.

The vasodilator effect on the microcirculation and the PPARs alpha have an insulin-like activity, mimicking the beneficial effects of sport.

Furthermore, it is advantageous, at the dietary level, to reduce the intake of saturated fats to the benefit of short-chain polyunsaturated (omega-3) fats or to enrich it. These omega-3 fats activate the chain of PPARs which will activate fat oxidation in the mitochondria of the cells of the liver, of adipose tissues and of muscle tissues. The PPARs induce the apoptosis of macrophages activated in turn by TNF alpha, in tissues with a high fatty acid catabolism, which include hormone-dependent fatty tissues. The TNFα factor is triggered when the adipocytes are hypertrophy, in particular during established obesity.

The sirtuin stimulators, like the STACs, combined with omega-3 fats, will also have an insulin-like effect and will mimic the lipolytic effects of sporting activity.

The effectiveness of the compositions is interdependent on the biological clock and on the oscillations thereof. A potentiation of the “therapeutic” effects is obtained if a chronomodulated plan is adhered to.

The process of the invention makes it possible to also overcome the technical problem consisting, according to a cellulo-score plan, in the indication of cellulite, or a chronomodulated scale of application or intake of the compositions in order to improve the effectiveness thereof, in the strict observance of the biological rhythm of the cell, totally autonomous of the central neurological clock (chronobiology rhythm): alternation of activity phase and resting phase (of 12 hours), concerning all types of cells.

The compositions could prove to be not very probative, or ineffective, or even harmful to human health, if long-term cell desynchronization was to be triggered, by taking no account of the moment of application or of oral intake of the composition, like with the taking of an anticancer medicament which could lose effectiveness depending on the time at which it is taken (cell in constant time shift). A chronobiological scale over a period of 24 hours makes it possible to indicate the zones suitable for application of the compositions, so as to obtain a peak optimization of the effectiveness of the active ingredients, according to a personalized lifestyle.

Advantageously, the compositions according to the present invention make it possible to treat the various types of cellulite: adipose, fibrous, or aqueous cellulite.

Some compositions combine stimulators of collagen production (neocollagen production). The proliferation of collagen fibers contributes to the production of interstitial tissue fibrosis, triggered by tissue hypoxy. The type of cellulite is never specified; however, it is impossible to obtain good effectiveness if the same composition is used for adipose, fibrous or edematous cellulite. The inflammatory component is not the same. Here again, it is possible to trigger degenerative processes, including cancer. Furthermore, in any stress process, a reaction is observed in which there is collagen fiber proliferation, which is not part of the desired approach. Poorly controlled stress is a provider of risks: cardiovascular risks, degenerative diseases and cancer. Thus, in the present invention, the addition of compounds which stimulate the formation of collagen fibers is avoided.

Furthermore, the present compositions make it possible to prevent inflammatory processes by reducing the negative effects of repeated stresses. It is known that aging brings about a time shift in the biological clock of cells. In this context, it is possible to propose a real protection and/or repair of the anti-inflammatory prevention mechanisms, by realigning the activity of the cell in its resting and working phases. It is acknowledged that chronic dephasing of the rhythm of life of these cells, desynchronization of the biological rhythm, causes a gradual slip (“time shift”) that can induce a loss for the cell of its ability to synthesize and its ability to eliminate its waste. Finally, this can result in a change in morphological configuration of the cell membranes with loss of recognition of the external medium, interrupting any mode of communication. Thus, the process of the invention makes it possible to combat this shift in cell rhythm and the consequences thereof.

Advantageously, the compositions according to the present invention, surprisingly, in particular in the presence of a slimming agent, make it possible to combat inflammatory processes (which include the cell aging phenomenon). Indeed, the activation of lipolysis, the size of the adipocytes, will make it possible, independently or in combination with vascular agents, to limit or block the glycation mechanism. These two mechanisms, possibly combined, will make it possible to combat most inflammatory mechanisms, whatever the type of tissue, in particular by blocking the pro-oxidizing mechanisms.

### DETAILED DESCRIPTION OF THE INVENTION

The process of the invention is in no way limited to the detailed description set out hereinafter, but makes it possible to illustrate the invention with examples of compositions and of use.

The kit of the invention contains two different compositions.

More specifically, the kit of cosmetic, pharmaceutical, food or veterinary compositions of the invention comprises a first composition containing at least one sirtuin inhibitor and at least one HSP inhibitor, and a second composition comprising at least one sirtuin activator and at least one HSP activator, the two compositions being packaged separately.

### First Composition

Typically, the first composition intended to be administered during the day, or diurnal composition, comprises a sirtuin inhibitor, an HSP inhibitor, and optionally a slimming agent and/or a microcirculation activator (also known as vascular activator).

Advantageously, the sirtuin inhibitor is chosen from tocopheryl nicotinate, niacin (also known as vitamin B3), sirtinol, cirsimarin, cirsimaritin, capsaicin, any one of the sirtuin inhibitors of formulae 1 to 25, 30 and 32-65 mentioned in patent application US 2005/0136537 by Sinclair et al., and any combination thereof. One of these molecules or a plant extract containing same can be incorporated into the first composition. A source of niacin will, for example, be an extract of mushroom, such as oyster mushroom. A source of capsaicin will, for example, be an extract of pepper, such as yellow pepper. An extract of black cumin (also known as nigella) also has sirtuin-inhibiting properties.

The sirtuin inhibitor can also be a plant extract containing one of these compounds. The sirtuin inhibitor is, for example, chosen from extracts of Micotea debilis, in particular those containing cirsimarin and/or cirsimaritin, and the proteins contained in whole cereals, such as peas, lentils, barley, wheat or rye, and the aqueous extracts of these cereals.

The sirtuin inhibitor can also be co-enzyme Q10 (ubiquinone).

Advantageously, the HSP inhibitor is chosen from deguelin, quercetin (also known as meletin, sophretin, or pentahydroxyflavone), L-glutamine or a plant extract containing same, myricetin, kaempferol, coumestrol, and any combination thereof.

The HSP inhibitor can also be chosen from plant extracts containing an HSP-inhibiting molecule, such as an extract of Sericea mundulea, an extract of red berries containing myricetin, an extract of onion which is a source of quercetin, an extract of caper which is a source of kaempferol, or an extract of soya which is a source of coumestrol.

Care will be taken not to incorporate into the first composition an ingredient that has a sirtuin-activated activity or an HSP-activating activity, so as not to abolish the effects produced by the sirtuin inhibitor and the HSP inhibitor that the first composition contains. The first composition is advantageously free of a compound which has a sirtuin-activating activity or an HSP-activating activity. This is not the case for compositions which have been described in the prior art, in particular the compositions containing a sirtuin inhibitor and an HSP inhibitor.

### Second Composition

Typically, the second composition intended to be applied at night, or nocturnal composition, comprises a sirtuin activator, an HSP activator, and optionally a slimming agent and/or a vascular activator (microcirculation activator).

Advantageously, the sirtuin activator is chosen from
a polyphenol such as trans-resveratrol or a resveratrol derivative, such as diphenyl resveratrol or dihydroresveratrol,
any one of the sirtuin activators mentioned in patent application US 2005/0136537 by Sinclair et al.,
FOXO 3, which is a transcription factor (Forkhead box subgroup O),
xanthohumol or a plant extract containing same, for example an extract of hops,
isoliquiritigenin or a plant extract containing same, for example an extract of liquorice,
phloridzin or a plant extract containing same, for example an extract of apple,
piceatannol or a plant extract containing same, for example an extract of rhubarb,
any one of the sirtuin activators mentioned in patent application WO 2007/104867, and
a natural flavonoid such as fisetin or a plant extract containing same, such as an extract of strawberries, of grape, of apple or of tomato. Fisetin improves the biochemical memorization pathways of neurons, is a powerful antioxidant, and combats neuronal apoptosis. It is part of the distinct class of STACs, released at the time fruit and/or vegetables are picked (picking stress). It appears to act via an allosteric mechanism, by causing an overrepresentation of Sir-2 homologs and by blocking cyclin-dependent protein kinases (CDK2 and CDK4). It furthermore appears to act on telomerases by protecting them against degradation and end-to-end fusions,
any combination thereof.

Advantageously, the HSP activator is chosen from
TEX-OE or an extract containing same, such as an extract of Barbary fig epicardium. It accelerates the appearance of HSPs (8 to 20 minutes after the application of TEX-OE, without there having been any stress,
verbascoside (phenolic agent) or a plant extract containing same, such as an extract of vervain or an extract of olive,
an extract of Ficus Opuntia indica fruit,
D-trehalose which can be extracted from a brown algae (laminarin) or from Ganoderma lucidum and which will act as a glaze protecting the cell membrane (cytoprotector), oxygen scavenger and antioxidant by blocking glycation; it makes it possible to combat turning rancid in the event of the addition of light-chain polyunsaturated fats, omega-3 fats,
rosavin or a plant extract containing same, such as an extract of Rhodiola rosea,
arginine or an extract containing same, for example an extract of walnut,
selenium, and
any combination thereof.

Rosavin or a plant extract containing same is advantageously combined with L-carnitine, in order to slow down adipogenesis.

Care will be taken not to incorporate into the second composition an ingredient that will have a sirtuin inhibiting activity or any HSP-inhibiting activity, so as not to abolish the effects produced by the sirtuin activator and the HSP activator that the second composition contains. The second composition is advantageously free of a compound which has a sirtuin-inhibiting activity or an HSP-inhibiting activity.

### Slimming Agent

Each of the two compositions can also contain at least one slimming agent, preferably a lipolysis-activating or adipogenesis-inhibiting slimming agent.

The term “slimming agent” is intended to mean an agent which has properties of inhibiting fat storage or of activating the release of stored fats contained in the adipocytes. The slimming agent can be chosen from slimming agents which act on various biological targets, in particular
by stimulating HSL (hormone-sensitive lipase), and/or
by stimulating beta 1/2 adrenergic receptors, and/or
by inhibiting LPL (lipoprotein lipase), and/or
by inhibiting alpha-2 adrenergic receptors, and/or
by blocking adenosine Ata receptors, and/or
by blocking cell differentiation into adipocytes by blocking the induction of peroxisome proliferator-activated receptors (PPARs gamma).

Advantageously, the slimming agent can be chosen from:
polyphenols, including tea epigallocathechin-3-gallates (ECGCs) or a green tea cathechin,
luteolin,
forskolin or a plant extract containing same, such as an extract of Coleus,
alpha-linoleic acid (ALA),
any combination thereof.

The composition is advantageously free of caffeine or contains same in a very small amount.

Adipogenesis inhibitors may be extracts of Garcinia, of Baccharis or of Hortinia.

When the composition contains several slimming agents, forskolin is preferably the dominant slimming agent by weight of the mixture.

Care will be taken not to incorporate into the first composition a slimming agent that will have, in addition to its actual slimming action, a sirtuin-activating activity or an HSP-activating activity, so as not to abolish the effects produced by the sirtuin inhibitor and the HSP inhibitor that the first composition contains.

Care will also be taken not to incorporate into the first composition a vascular activating agent that will have, in addition to its actual blood circulation-activating action, a sirtuin-activating activity or an HSP-inhibiting activity, so as not to abolish the effects produced by the sirtuin inhibitor and the HSP inhibitor that the first composition contains.

### Vascular Activating Agent

Each of the two compositions can also contain at least one vascular activator, preferably a microcirculation activating agent.

According to one preferred embodiment, the compositions of the invention make it possible to take into account the fundamental component represented by the activation of the vascular system as a factor mimicking physical activity, triggering lipomobilization, activating lipolysis mechanisms (distinction between direct and indirect lipolysis according to the receptors activated or inhibited). The activation of the vascular component will contribute to accelerating fat wasting, by mimicking the beneficial effect of sport. The microcirculation activator can, for example, act by blocking the differentiation of the cells of the vascular stroma into adipocytes, by causing blocking of glycation phenomena, by accelerating lipomobilization, or by stimulating peroxisome proliferator-activated receptors (PPARs alpha).

The oxygen enrichment of a product can trigger an overproduction of inflammation pro-activators. The oxygen content of a cell at night decreases and is physiologically less than 5%; however, if the state of the vascular distribution network is insufficient, in a period of stress, the levels of reactive oxygen species (ROSs) can increase considerably and cause substantial cell damage. The tissue oxygenation will be too low to repair the damage caused by the hypoxia and will accelerate the accumulation of oxidative derivatives. The hypoxia will consequently lead to a blocking of adenosine triphosphate (ATP) production and the appearance of tissue fibrosis. However, the principle of the invention is to increase ATP production by the cell in order to burn storage fats, while at the same time preserving its resources as well as possible. However, the lipolysis mechanism does not only deal solely with adipocyte cells and is not sufficient to explain all the mechanisms of fat wasting.

Obesity or excess weight is associated with an increase in circulating levels of adipokines (LPL, leptin, adiponectin) and of cytokines (TNF alpha, IL-Beta, IL-6), which are activators of inflammatory processes, and leads to the mobilization and differentiation of certain cells (monocytes, mesenchymical cells) into adipocytes. The stromal vascular fraction (SVF) contains a pool of cells capable of differentiating into adipocytes or into endothelial cells; vasodilatation of the microcirculatory system will contribute to reducing the risks thereof.

Increasing the blood flow rate of a tissue will concomitantly contribute to reducing the risks of insulin resistance and to accelerating lipomobilization. Any activation of the vascular system is likened in terms of its effects to those produced by physical activity, which encompasses voluntary or involuntary activities (NEAT: non-exercise activity thermogenosis), such as standing up for an extended period of time, maintaining a pose, imperceptible movements relating to nervousness: a “sport-like” effect. The activation of the microcirculation will trigger the activation of heat shock proteins (HSPs) and that of peroxisome proliferator-activated receptors alpha (PPARs). The agents increasing microcirculation have an insulin-like activity, mimicking the beneficial action of a sporting activity, considered to be a positive stress.

Advantageously, the vascular activator is chosen from AHAs (alpha-hydroxy acids), and isoflavones or a plant extract containing same, such as an extract of cassova or an extract of clover. Among the isoflavones, mention may be made of genistein. The microcirculation activator can be chosen from an extract of St. John's wort, an extract of ginkgo biloba, an extract of Sophora japonica, an extract of Centella asiatica, an extract of ruscus (Ruscus aculeatus), an extract of climbing ivy, an extract of agrimony, an extract of mouse-ear hawkweed (Hieracium pilosella), an extract of sweet clover (Melilotus officinalis), an extract of beech buds, an extract of horse chestnut, an extract of dermochlorella, rosavin or a plant extract containing same, such as an extract of Rhodiola rosea plant, and any combination thereof.

A combination of the abovementioned microcirculation activators may be incorporated into each of the compositions.

Care will be taken not to incorporate into the second composition a slimming agent that will have, in addition to its actual slimming action, a sirtuin-inhibiting activity or an HSP-inhibiting activity, so as not to abolish the effects produced by the sirtuin activator and the HSP activator that the second composition contains.

Care will also be taken not to incorporate into the second composition a vascular activator that will have, in addition to its actual blood circulation-activating action, a sirtuin-inhibiting action or an HSP-inhibiting action, so as not to abolish the effects produced by the sirtuin activator and the HSP activator that the second composition contains.

The lipolysis activators and the microcirculation activators will be used at different concentrations in the diurnal composition and in the nocturnal composition, and those skilled in the art will be capable of adjusting these concentrations according to the desired results.

To give examples of particular dosages for the preferred activators.

According to one embodiment, the day composition contains deguelin, optionally in the form of an extract of Sericea mundulea, as HSP inhibitor, optionally combined with an extract of ginkgo biloba and/or with an extract of St. John's wort.

An example of a composition intended to be administered during the day which is preferred according to the invention contains, for 100 ml of composition:

at least one sirtuin inhibitor chosen from:

| | |
|---|---|
| Tocopheryl nicotinate | from 0.001 mg to 1 g |
| Niacin | from 0.001 mg to 1 g |
| Sirtinol | from 0.001 mg to 1 g |
| Aqueous extracts of whole cereals | from 0.001 mg to 1 g of dry |
| | extract |
| Capsaicin | from 0.001 mg to 1 g |
| Co-enzyme Q10 (ubiquinone) | from 0.001 mg to 1 g |
| and mixtures thereof, | |

at least one HSP inhibitor chosen from:

| | |
|---|---|
| Quercetin | from 0.001 mg to 3 g |
| Deguelin | from 0.001 mg to 1 g |
| Rice peptide (L-glutamine) | from 0.001 mg to 1 g |
| and mixtures thereof | |

at least one lipolysis activator chosen from:

| | |
|---|---|
| Forskolin | from 0.001 mg to 1 g |
| Green tea cathechin | from 0.001 mg to 1 g |
| Luteolin | from 0.001 mg to 1 g |
| Alpha-linoleic acid | from 0.01 mg to 1 g |
| and mixtures thereof | |

and at least one circulation activator chosen from:

| | |
|---|---|
| Genistein | from 0.001 mg to 1 g |
| Ginkgo biloba | from 0.001 mg to 1 g |
| Dermochlorella | from 0.001 mg to 1 g |
| Sweet clover | from 0.001 mg to 1 g |
| Horse chestnut | from 0.001 mg to 1 g |
| Arnica | from 0.001 mg to 1 g |
| Witch hazel water | from 0.001 mg to 1 g |
| and mixtures thereof. | |

An example of a composition intended to be administered at night which is preferred according to the invention contains, for 100 ml of composition:

at least one sirtuin activator chosen from:

| | |
|---|---|
| Di hydroresveratrol | 0.01 g |
| Fisetin (dehydrated strawberries) | 0.001 g |
| FOXO 3 | 0.001 mg to 1 g |
| and mixtures thereof, | |

at least one HSP activator chosen from:

| | |
|---|---|
| TEX-OE | 0.001 mg to 1 g |
| D-Trehalose | 0.001 mg to 1 g |
| Rosavin | 0.001 mg to 1 g |
| Arginine | 0.001 mg to 1 g |
| and mixtures thereof, | |

at least one lipolysis activator chosen from:

| | |
|---|---|
| Forskolin | from 0.001 mg to 1 g |
| Green tea cathechin | from 0.001 mg to 1 g |
| Luteolin | from 0.001 mg to 1 g |
| Alpha-linoleic acid | from 0.01 mg to 1 g |
| and mixtures thereof, | |

and at least one circulation activator chosen from:

| | |
|---|---|
| St. John's wort | from 0.001 mg to 1 g |
| Ginkgo biloba | from 0.001 mg to 1 g |
| and mixtures thereof. | |

Other ingredients of the compositions

Antioxidants such as whey protein isolates (glutathione), retinol, curcumin combined with piperine, zinc, derivatives of B-group vitamins other than vitamin B3, vitamin C or an omega-3 fatty acid, and any combination thereof, can be incorporated into the diurnal and nocturnal compositions.

Vitamin C may only be used in the diurnal preparations.

In order to sweeten the preparations of the oral compositions and to avoid the risks of hypoglycemic attacks, substances with a low glycemic index, such as stevia, xylitol, or beta-stirol, will be chosen.

Concentrates of water-soluble or concentrated tannins may be advantageous as oxygen free radical scavengers, heavy metal scavengers and sugar oxoreduction agents, and for reducing the rancid taste caused by the addition of fats (omega-3) to compositions administered orally.

The compositions according to the process of the present invention advantageously act on cells, and in particular adipocytes, by limiting inflammatory processes.

Advantageously, the compositions of the present invention are cosmetic or pharmaceutical compositions intended for topical application. The term “topical application”, used here, means bringing into contact with the surface of the skin. The compositions for topical application can advantageously be combined with compositions administered orally so as to complete or reinforce the action thereof.

The compositions may be in the form of a beverage, a food sauce or a nutraceutical composition, such as capsules, tablets or a powder to be diluted.

Advantageously, the compositions of the invention are formulated in a form chosen from the group consisting of an aqueous or oily solution, in particular in the form of beverages or syrups, an aqueous or oily cream or gel; a milk; an oil; a mask; a simple or multiple emulsion, a microemulsion or a nanoemulsion, which is in particular oil-in-water or water-in-oil or multiple; a lotion; a liquid soap; a spray formulation; a paste; a dermatological bar; an ointment; a solid, in particular in granule, tablet, powder, vial or pencil form; or a foam.

A cosmetic composition according to the invention preferably contains at least one excipient chosen from the group consisting of preservatives, emollients, emulsifiers, surfactants, moisturizers, thickeners, conditioners, matting agents, stabilizers, antioxidants, texturing agents, gloss agents, film-forming agents, solubilizers, pigments, dyes, fragrances and sunscreens. The compositions of the invention can in particular contain the following ingredients: purified water, purcellin oil, paraffin, Sepigel 305, plant glycerol (moisturizer), Aloe vera, cetyl alcohol, dodecanol (emollient-surfactant), tocopheryl acetate (Vit. E and derivatives), silicone oil, retinoic acid (synthesis of dermal fibers), extract of thyme (antiseptic), extract of camomile (cleanser), zeaxanthin (natural antioxidant), hyaluronic acid (to compensate for the fat wasting and avoid slackening), beta-sitosterol (lowers cholesterol/anti-inflammatory/anticancer agent).

Dyes, preservatives and/or stabilizers (Carbopol® 981), preferably chosen from natural products, can also be added.

These compositions may be colored or colorless, just like their fragrance can be incorporated during their production or afterwards, by providing a kit comprising a fragrance that will be selected (a few drops to be mixed in).

Advantageously, the compositions have the effect of reducing the risks of glycation. Glycation is one of the phenomena responsible for vascular aging, and for the occurrence of cell damage, which can trigger the release of cytokines.

The proliferation of collagen and/or elastin and/or fibrin fibers contributes to the worsening of tissue hypoxia by compression of the microvessels of the tissue, and to the occurrence of tissue fibrosis, and promotes the occurrence of cellulite.

Advantageously, the active ingredients of the compositions, according to the present invention, avoid such proliferations.

### Galenics Suitable for the Various Types of Cellulite

The kit of compositions according to the invention makes it possible to combat cellulite. Cellulite is an inflammatory phenomenon responsible for the fatty acid storage by adipocytes, for the differentiation of SVF cells into adipocytes, for tissue fibrosis via glycation and for the decrease or disappearance of the microcirculatory network of tissues or organs. Obesity is characterized by modifications of the metabolic and secretory pathways of adipocytes. It is synonymous with a chronic inflammatory phase. Activating lipolysis makes it possible to prevent the cell differentiation of pre-adipocytes into adipocytes and of the cells of the stromal vascular fraction.

By virtue of the compositions of the invention, lipolysis corresponds to one of the treatments for the inflammation.

For the three types of cellulite, additions of different active molecules are made, as appropriate, and the galenics for carrying these active molecules are advantageously adjusted.

A typical composition for combating adipose cellulite is a water-in-oil emulsion.

A typical composition for combating fibrous cellulite is a gel or a cream in the form of an oil-in-water emulsion.

A typical composition for combating aqueous cellulite is a cream in the form of an oil in water.

The inflammatory component is not the same depending on the steps of adipocyte differentiation. It is divided up into three steps: adipoblast proliferation, pre-adipocytes which have acquired sufficient nuclear receptors for adipogenesis (PPARs, RXR), and the lipid storage phase in the adipocyte. However, any forced expression of nuclear receptors, including PPARs gamma 2, in a fibroblast can induce its differentiation into an adipocyte. This explains the importance of defining the inflammatory state of a tissue, for instance of a subcutaneous cellulite zone.

It is understood that adipose tissue has the status of a central organ for metabolism and inflammation, and not only a storage status. It is acknowledged that adipose tissue contains not only adipocytes, but other cells which explain its endocrine activity.

As a result, the same composition will not be used for adipose cellulite, fibrous cellulite or edematous cellulite, hence the need to adjust the galenic of the compositions, but while combining them with a vascular factor that will reinforce the direct or indirect lipolysis of the tissues (blocking of beta1/alpha2-adrenergic receptors, of adenosine Ata receptors and of adipogenesis by blocking peroxisome proliferator-activated receptors gamma—PPARs), while at the same time reducing inflammatory phenomena.

The activation of sirtuin genes (Sirt1) contributes to lipolysis by blocking PPAR factors which stimulate the transcription of several genes in favor of adipogenesis. Sirtuins, by mimicking calorie control, will maintain respiratory metabolism (blocking of UCP 2 permease), thus preserving adenosine triphosphate (ATP) production. Nicotinamide adenine dinucleotide (NAD) reinforces the activity of the enzymes of the sirtuin family which have an action on certain anti-inflammatory processes.

Synergy between the cells of the tissues involved in metabolism and the inflammatory cells is underlined.

The composition according to the present invention can be used in the context of a method for slimming cosmetic care, such as a method for slimming care using a machine such as a laser, a radiation device or a mechanical skin massaging device.

The products of the invention are intended both for female care and for male care.

The present invention covers a method for slimming care and/or for prevention of inflammatory processes (in particular cosmetic, dermocosmetic, pharmaceutical or veterinary care, via the topical and/or oral route): comprising the application or the intake, in the morning or when getting up, of a diurnal composition according to the process of the present invention and the application or intake, in the evening or when going to bed, of a nocturnal composition according to the present invention.

A subject of the present invention is also the kit of compositions previously described for use in the treatment and/or prevention of inflammatory processes in human beings or in animals, such as excess weight, obesity, type 2 diabetes, cardiovascular diseases, cancer or skin aging.

All of the abovementioned compositions comprising sirtuin and HSP inhibiting pairings or sirtuin and HSP activator pairings, and preferably applied according to the alternation of the nycthemeral cycle, are compositions termed “front-line treatment”, for obtaining maximum effectiveness on the cells. This treatment typically lasts 30 days.

It is preferably followed by a “maintenance” treatment which typically lasts 30 days. By virtue of this maintenance treatment combined with the front-line treatment, the method according to the present invention is all the more advantageous.

Thus, the present invention covers compositions comprising at least one sirtuin modulator (inhibitor or activator) combined with at least one HSP modulator (inhibitor or activator), optionally in the presence of at least one slimming agent and/or at least one vascular activating agent.

The present invention also covers the use of a combination of at least one sirtuin modulator and of at least one HSP modulator as active ingredients, in a slimming or inflammatory-process-preventing composition.

A subject of the invention is also a cosmetic, pharmaceutical, food or veterinary composition comprising at least one HSP activator and at least one sirtuin activator, as previously described. This composition can have all the characteristics described in relation to the second composition of the kit previously described. The invention also relates to the use of such a composition for increasing the activity against cellulite and inflammatory processes of a composition containing at least one sirtuin inhibitor and at least one HSP inhibitor.

Advantageously, the compositions can comply with the cosmetic directives of the EEC (Annex VI: 76/768/EEC), in order to obtain the bio-cosmetology or biological oral products label.

The methods for evaluating the effect of the present invention on cellulite can be chosen from:

1) Profilometry: digital photos

2) Echography at 2 MHz: to verify hypodermal invaginations, thickness of the adipose panicle.

3) Cutometric measurements of skin firmness (firming capacity): vacuum pump, which by suctioning calculates cutaneous resistance and the capacity thereof to return to its original state (elasticity).

4) Microangioscopy (400-fold magnification): evaluates capillary tortuosity, pericapillary edema and dilation of the venular side.

5) Simple blood tests for the level of inflammation: C-reactive protein (CRP) and albumin (Michaud D S, Liu S, et al. ‘Dietary glycemic load’, Cancer Epidemiology, Biomarkers & Prevention, 2005; 14(1): 138-47)
albumin>35 g/l or CRP<10 mg/d: minimal risk;
albumin<35 g/l or CRP>10 mg/l: medium risk;
albumin<35 g/l and CRP>10 mg/l: high risk.

These tests make it possible to observe the beneficial effect of the combination of the compositions according to the present invention in comparison with the prior art compositions used alone, in particular those comprising sirtuin activators.

The compositions are preferably applied or ingested for at least 30 days, twice a day (morning and evening) according to a chronomodulated plan.

The present invention covers in particular a method for care comprising the diurnal application of a diurnal composition according to the present invention, and/or the nocturnal application of a nocturnal composition according to the present invention. The diurnal/nocturnal cycle is assessed according to the hours of the day, taking into account the time zone and when the individual subject to the care gets up and goes to bed. The cycle can generally vary by two to three hours depending on individuals and their lifestyle.

Biological rhythms affect cell metabolisms, the major vital functions, the nervous system and motricity. 24-hour rhythms are usually synchronized with day-night alternation, activities and rest.

The adaptation of organisms to the cyclic variations of the environment (seasons, day/night alternation, tide cycle, lunar cycle) make it possible to synchronize body, physiological and behavioral changes with environmental changes.

The cells have a circadian rhythm autonomy; they can synchronize their activity, and can constitute one or more individualized structures called oscillators (or biological clock).

Biological rhythms are generally predetermined, and modulated by synchronization factors.

The chronotype classifies individuals mainly according to their preference for rising early (morning types) or going to bed late (evening types), compared with the general population. This preference has been associated with a circadian phase that is respectively earlier or later.

The sleep/wake cycle depends on the circadian rhythm of the central nervous system (CNS) through the secretion of cortisol: the maximum blood load of glucocorticoids precedes the end of the night by 2 h, and very probably guarantees neoglucogenesis of protein origin at the time of awakening.

An external desynchronization occurs: during transmeridian flights, working at night or work which began before the normal time of awakening (maximum secretion of cortisol around 7-8 am).

The secretion of melatonin (sleep hormone) will itself also be shifted in the event of desynchronization of the organism.

Following an external desynchronization, the circadian temporal structure will be resynchronized according to new time constraints. When there is an 8-hour phase advance (time difference of 8 hours), if a physical activity is performed for a period of more than 2 hours, the resynchronization takes place in less than 48 hours, whereas the normal time would have been several days.

By mimicking a physical activity, through the oral intake of the slimming and/or inflammatory-process-preventing compositions, or through the application of these same compositions in the skin, it is possible to obtain a resynchronization effect on the circadian rhythm of the central nervous system.

The modification of the activity/rest cycle appears to be one of the most relevant markers. This modification is easily measured using small piezoelectric accelerometers worn on the wrist (in a human being) for at least three days.

The compositions may be applied or taken orally in a shifted manner, according to the external desynchronizations (adjuster scale envisaged).

It may, for example, be envisioned to apply or take the day composition between 6-9 am and the night composition between 6 pm and 9 pm.

For individuals who get up later, it may be envisioned to apply or take the day composition between 9-11 am and the night composition between 10 pm and midnight.

In the event of an occasional shift: it will be recommended not to take the compositions and to stop for a day.

In the event of the night composition or the day composition being forgotten, it is recommended to begin again only the following evening, and stop the treatment for 24 hours.

The adjuster scale efficiently shifts the oral intake or the application, according to night work or day work.

The risk factors for cell desynchronization will be avoided, among which mention may be made of alcohol, cola-based beverages, coffee, tea, beta-blockers, glucocorticoids, nicotine, tobacco, light during the night, jet lag, a lack of physical activity; and chronic stress (working at night or early in the morning).

It is recommended to compensate for the deficiencies by taking in: taurine, magnesium transporter, B6 vitamins and omega-3 fats for stimulating melatonin secretion; it is possible, through the oral intake of tryptophan, to increase the secretion of N-acetylcysteine (NAC), via serotonin production. Among the sources of tryptophan, mention may be made of dry fruits (almonds, walnuts, hazelnuts, etc.), crucifers and legumins (B6, Mg), avocado (B6), magnesium-rich water, fatty or oily fish rich in omega-3, white meat, food supplements containing Mg-taurine, B6 and omega-3.

A subject of the invention is also the use of the combination of the diurnal and nocturnal compositions previously described for obtaining a resynchronization effect on the circadian rhythm of the central nervous system.

Other purposes, characteristics and advantages of the process of the invention will become clearly apparent to those skilled in the art following the reading of the explanatory description which refers to examples that are given only by way of illustration and that could not in any way limit the scope of the invention.

The examples are an integral part of the present invention and any characteristic which appears to be novel over any prior art on the basis of the description taken as a whole, including the examples, is an integral part of the invention in terms of its function and its generality. Thus, each example has a general scope.

Furthermore, in the examples, all the percentages are given by weight, unless otherwise indicated, and the temperature is expressed in degrees Celsius unless otherwise indicated, and the pressure is atmospheric pressure, unless otherwise indicated.

### EXAMPLES

The amounts are given simply for illustrative purposes and are understood to be for compositions of 100 ml.

The amounts can be modified by those skilled in the art and can be easily determined by those skilled in the art.

### Example 1

### Slimming Day Composition

A diurnal composition of 100 ml may contain the following ingredients:

Sirtuin Inhibitors:

| | |
|---|---|
| Tocopheryl nicotinate | 0.01 g |
| Niacin | 0.004 g |
| Sirtinol | 25 micromol |
| Aqueous extracts of whole cereals | from 0.001 mg to 1 g of |
| | dry extract |
| Capsaicin | from 0.001 mg to 1 g |
| Co-enzyme Q10 (ubiquinone) | 0.15 g |

HSP Inhibitors:

| | |
|---|---|
| Quercetin | 0.025 g |
| Deguelin | 0.001 g |
| Rice peptide (L-glutamine) | from 0.001 mg to 1 g |

Lipolysis Activators:

| | |
|---|---|
| Forskolin | from 0.001 mg to 1 g |
| Green tea cathechin | from 0.001 mg to 1 g |
| Luteolin | from 0.001 mg to 1 g |
| Alpha-linoleic acid | 0.08 g |

Circulation Activators:

| | |
|---|---|
| Genistein | 0.02 g |
| Ginkgo biloba | from 0.001 mg to 1 g |
| Dermochlorella | from 0.001 mg to 1 g |
| Sweet clover | from 0.001 mg to 1 g |
| Horse chestnut | from 0.001 mg to 1 g |
| Arnica | from 0.001 mg to 1 g |
| Witch hazel water | from 0.001 mg to 1 g |

Other compounds may be added for their antioxidizing action (glutathione), or anti-inflammatory action, or for blocking the risks of angiogenesis (curcumin or piperine).

### Example 2

### Slimming Night Composition

A nocturnal composition of 100 ml may contain the following ingredients.

Sirtuin Activators:

| | |
|---|---|
| Dihydroresveratrol | 0.01 g |
| Fisetin (dehydrated strawberries) | 0.001 g |
| FOXO 3 | 0.001 mg to 1 g |

HSP Activators:

| | |
|---|---|
| TEX-OE | 0.001 mg to 1 g |
| D-Trehalose | 0.001 mg to 1 g |
| Rosavin | 0.001 mg to 1 g |
| Arginine | 0.001 mg to 1 g |
| Selenium | from 0.001 mg to 1 g |

Circulation Activators:

| | |
|---|---|
| St. John's wort | from 0.001 mg to 1 g |
| Ginkgo biloba | from 0.001 mg to 1 g |

Lipolysis Activators:

| | |
|---|---|
| Forskolin | from 0.001 mg to 1 g |
| Green tea cathechin | from 0.001 mg to 1 g |
| Luteolin | from 0.001 mg to 1 g |
| Alpha-linoleic acid | from 0.01 mg to 1 g |

### Example 3

### Kit Comprising a Day Composition and a Night Composition for Combating Adipose Cellulite

Each composition has a volume of 100 ml.

3.1 Day Composition

The following activators are added to the composition of example 1:

| | |
|---|---|
| Purcellin oil/wheat germs | 0.001 mg to 1 g |
| Hyaluronic acid | 0.001 mg to 1 g |
| Adipogenesis-inhibiting active agents | 0.001 mg to 1 g |

3.2. Night Composition:

The following active agents are added to the composition of example 2:

| | |
|---|---|
| Alpha-linoleic acid | 0.001 mg to 2 g |
| Purcellin oil/wheat germs | 0.001 mg to 1 g |
| Hyaluronic acid | 0.001 mg to 1 g |
| Catechin: | 0.01 mg to 1 g |
| Adipogenesis-inhibiting active agents | 0.001 mg to 1 g |
| L-carnitine (fat burner/stimulates appetite) | from 0.01 mg to 1 g |

Each of the compositions can be in the form of a water-in-oil or oil-in-water-in-oil emulsion.

### Example 4

### Kit Comprising a Day Composition and a Night Composition for Combating Fibrous Cellulite

Each composition has a volume of 100 ml. Each compound is included in a proportion of from 0.01 mg to 1 g.

4.1. Day Composition

The following active agents are added to the composition of example 1:
Selenium
Centella asiatica
Wheat protein
Fruit acids
Retinoic acid (apoptosis)
Organic silicon
Dextran sulfate

4.2. Night Composition:

The following active agents are added to the composition of example 2:
Selenium
Centella asiatica
Wheat protein
Fruit acids
Organic silicon
Dextran sulfate
Grape OPC (anti-glutathione active agent)
Cocktail of red fruits: blackcurrants-blueberries-strawberries-raspberries-bilberries
   Each of the compositions can be in the form of an oil-in-water or water-in-oil-in-water emulsion.

### Example 5

### Kit of Compositions for Combating Aqueous Cellulite

The compositions of example 3, in which the draining agents and the hyaluronic acid are increased in concentration, are taken. Glycerol (humectant for facilitating penetration) and extracts of Agrimony, of mouse-ear hawkweed and of Ruscus are additionally added to each composition.

Each of the compositions can be in the form of an oil-in-water or water-in-oil-in-water emulsion.

### Example 6

### Compact Day and Night Powders for any Type of Skin

Day Powder Comprising:
trehalose;
niacin;
silicon;
urukum (provitamin A and selenium);
laminarin alginate;
calcium (Undaria prunatifida);
wheat protein powder
lycopene, zeaxanthin (carotinoids)

Night Powder Comprising:
vitamin E/vitamin A/selenium;
fisetin;
dihydroresveratrol;
silicon;
urukum (provitamin A and selenium);
laminarin alginate;
calcium (Undaria prunatifida);
wheat protein powder
lycopene, zeaxanthin (carotinoids)

The expected beneficial effect of the inflammatory mechanism-preventing effect will be an improvement in the general condition (sensation of feeling better, decrease in sensitivity to stresses, mild analgesia effect), a more satisfactory general appearance and an improved effect in relation to the use of sirtuin modulators alone.

### Example 7

### Day Beverage for Sportsmen and Sportswomen

Each compound is present in an amount of from 0.01 mg to 1 g.
Extract of urukum essentially and of acerola, containing flavonoids;
polyphenols and flavonoids derived from: dehydrated strawberries (40%), raspberries (20%), blueberries (20%), acai berries (20%);
a moisturizing agent such as a pure aloe vera juice;
a probiotic agent (nonobligatory) (in order to treat functional colitis or for reinforcing the defenses of the digestive mucosa, when there is no familial contraindication of a digestive cancer);
alfalfa proteins (vegetable proteins) and an extract of guarana (appetite-suppressant effect);
tryptophan (niacin precursor/P53 gene activator);
a policosanol (slimming agent by virtue of its fat-absorption-limiting action);
a sugar (stevia, trehalose);
a compound of the omega-3 type;
co-enzyme Q10;
alpha-linoleic acid;
glutathione; and
trace elements such as NaCI, Mg and/or K.

### Example 8

### Day Beverage for Premenopausal Women

Each compound is present in an amount of from 0.01 mg to 1 g.
Extract of urukum essentially and of acerola, containing flavonoids;
polyphenols and flavonoids derived from: dehydrated strawberries (40%), raspberries (20%), blueberries (20%), acai berries (20%);
a moisturizing agent such as a pure aloe vera juice;
a probiotic agent (nonobligatory) (in order to treat functional colitis or for reinforcing the defenses of the digestive mucosa, when there is no familial contraindication of digestive cancer);
alfalfa proteins (vegetable proteins) and an extract of guarana (appetite-suppressant effect);
tryptophan (niacin precursor/P53 gene activator);
a policosanol (slimming agent by virtue of its fat-absorption-limiting action);
a sugar (stevia, trehalose);
a compound of the omega-3 type,
an extract of St. John's wort,
borage and evening primrose oil (draining and soothing agents),
soya proteins for equilibrating the phytoestrogen ratio (if there is no gynecologist cancer contraindication).

### Example 9

### Anti-Inflammatory Preventive Evening Beverage

Each compound is present in an amount of from 0.01 mg to 1 g.
dihydroresveratrol;
fisetin;
polyphenols and flavonoids derived from: dehydrated strawberries (40%), raspberries (20%), blueberries (20%), acai berries (20%);
a moisturizing agent such as a pure aloe vera juice;
a probiotic agent (nonobligatory) (in order to treat functional colitis or for reinforcing the defenses of the digestive mucosa, when there is no familial contraindication of digestive cancer);
alfalfa proteins (vegetable proteins) and an extract of guarana (appetite-suppressant effect);
TEX-OE;
arginine;
a policosanol (slimming agent by virtue of its fat-absorption-limiting action);
a sugar (stevia, trehalose, etc);
an extract of St. Johns wort;
a compound of the omega-3 type.

### Example 10

### Beverage Kit and Clinical Study

The clinical tests were carried out on volunteer patients (10 to 20) who were not undergoing a treatment (no hormonal treatment, inter alia), who had no established diseases, and who had no family history of digestive and/or hormonal cancers.

The study consisted in comparing orally administered compositions:
a composition for group number 1 (G1), intended to be absorbed twice a day, containing sirtuin activators and an HSP activator;
a kit of the present invention, for group number 2 (G2), comprising a first composition based on ingredients chosen for their sirtuin-antagonist and HSP-antagonist effects, intended for consumption during the day, corresponding to the formula below, and a second composition containing sirtuin agonists and HSP agonists for absorption during the nocturnal period, corresponding to the formula below.

### Population Selected

The distribution of the patients recruited was not randomized, but was done on the basis of a mode introducing certain random parameters such as the timing of the recruitment, just as the patients happen to be seen in consultations, and the assignment of the treatment to be administered according to an equal number of individuals entering the trial.

The average age of the volunteers in the trial was 45 (+/−5).

The consumption of plants in the two groups was less than 3 to 5 fruits and vegetables per week, with a variant of (+/−3), which corresponds to an average consumption in a population with an age of 45 (+/−5) which is lower than the recommendations in force.

### The Formulae

For the products of group G1, rich in polyphenols and flavonoids, a period of 4 weeks of treatment was instituted, for better management. Two intakes per day were envisaged, more than 8 hours apart.

### I. Composition Administered to Group 1 Twice a Day

Extract of coconut water: 25 ml
Red fruit concentrate: 15 ml (sirtuin activator)
Aqueous extracts of apple: 15 ml (sirtuin activator)
Selenium 13 μg (HSP activator)
Caffeine: 1.5 ml (lipolytic agent)
Extract of pineapple (lipolytic agent)

### Guarana: 340 mg

### Whey: 25 ml

Pomegranate juice: 14 ml
Extracts of sweet potato: 2.5 g

### Carbomer 0.12 g

Preservative (E200—sorbic acid—natural) 0.65 g
Zinc (15% of recommended daily intake)

### Sodium 30 mg

Demineralized water QS for 250 ml

For the formulae given to group G2, one intake in the morning (at a fixed time with a flexibility of +/−1 hour) and one intake during the nocturnal period after 6 pm (correspondence between the decrease in circulating cortisol level and the change in cell activities), still at a fixed time, with a possible difference of at most one to two hours.

### II. Composition Administered to Group 2 During the Day

This formula contains several plant extracts in addition to the sirtuin inhibitors and HSP inhibitors. These extracts have different activities which combine together to have anti-apoptotic, anti-inflammatory, lipolytic and vasodilator effects.

Extract of yellow pepper: 10 mg
Extract of black cumin or nigella: 10 mg
Extract of Micotea debilis: 1 g
Aqueous extract of oyster mushroom: 10 ml
Aqueous extract of cassava root: 1.5 ml

### Quercetin 2.0 g

### Curcumin: 2.5 g

### Piperine: 20 ml

Unfermented green tea: 180 mg of ECGC
Extract of crucifers: 1.5 g
Aqueous extract of celery: 75 ml

### Carbomer 0.12 g

Preservative (E200—sorbic acid—natural) 0.65 g
Demineralized water QS for 60 ml

### III. Composition Administered to Group 2 at Night

Japanese knotweed rhizomes: 9.74 mg (rich in trans-resveratrol, sirtuin activator)
Powdered strawberry concentrate: 4 g (sirtuin activator)
Extract of rhubarb, source of piceatannol: 3 g (sirtuin activator)
Extract of liquorice, source of isoliquiritigenin: 1.5 g (sirtuin activator)
Extract of Barbary fig epicardium: 180 ml (HSP activator)
Brown algae laminarin powder: 3 g (HSP activator)
Aqueous extract of the plant Rhodiola rosea: 150 mg (HSP activator)
Extract of Ganoderma lucidum (containing HSP-activating trehalose)
Aqueous extract of unfermented green tea: 180 mg
Extracts of walnut: 2 g (rich in arginine and in omega-3)

### Carbomer 0.12 g

Preservative (E200—sorbic acid—natural) 0.65 g
Demineralized water QS for 60 ml

### Indications:

The indications were envisaged only as symptomatic treatment for improving the quality of life, the wasting of fat tissues (loss of volume and not of weight), and improving sleep quality. Since the diffusion of the active ingredients is systemic, it is envisaged to continue the experiment under several cycles.

Aging phenomena could not be evaluated owing to the route of absorption and the duration of the experiment, which was too short.

The same is true for the evaluation of the three types of cellulite, which would have required application for several months with testing of the subcutaneous tissues by echography.

The indications follow naturally from the potential for stress prevention, by calculating well-being, evaluated according to a scale ranging from 0 to 10, identical to those known for evaluating pain (VAS), sleep quality and appetite (body mass index/level of albumin and CRP (C-reactive protein).

Materials and methods:

### Fat Wasting:

Measurement of abdominal perimeter, and skin fold at the level of the brachial triceps.

### Blood Assays for Markers of Oxidative Stress:

The study made it possible, by means of comparative assays of the compositions G1/G2, to measure the production of oxygen-containing radicals (ROSs), reactive oxygen species produced as normal by-products of cell metabolism.

The nature of the modification of the proteins can provide important information on the type of oxidizing agent involved in the oxidation process.

The assays were carried out by various means, such as enzyme-linked spectrophotometric assay (ELISA), and/or by two-dimensional electrophoresis followed by immunoassay (Western blotting).

The interpretation of the oxidative stress tests proposed, for the purpose of evaluating the organism's inflammatory risk and the harmful consequences thereof, was imperatively based on a particularly careful treatment of the blood samples (preserved at −4° C.). The vast majority of the assays proposed for evaluating oxidative stress are in fact very sensitive to the conditions for preserving the samples (type of anticoagulant, storage temperature −20° C.).

The nature of the modification of the proteins can provide important information on the type of oxidizing agent involved in the oxidation process.

Generally, oxidative stress is defined as being the result of an imbalance between the balance of pro-oxidizing agents and defense systems (antioxidants), with, as a consequence, the production of active oxygen species (AOSs) responsible for damage to the cell which is often irreversible.

### Investigation of Oxidative Stress Damage

1—Lipid peroxidation: malondialdehyde (MDA), MTBARS and oxidized LDLs
2—DNA/RNA damage: 8-hydroxyguanosine (8 OHG) and thiol proteins
3—Oxidized proteins: Protein Carbonyl Content (PCC)
4—Antioxidants: oxidized glutathione (GSS) and reduced glutathione (GPSS), Superoxide Dismutase (SOD), Oxygen Radical Antioxidant Capacity (ORAC) and Total Antioxidant Capacity (TAC)

### 5—Antioxidants

### 6—Zinc

7—Sirtuin activities

### 1/Lipid Peroxidation:

The assaying of MDA represents only a small percentage (1%) of the lipid peroxide decomposition process, which makes it a marker of little interest. It was discarded from the test.

The immunological methods were chosen for assaying the oxidized LDLs.

The following were chosen for assaying:
Vitamin E, which has the ability to infiltrate into the fatty acids of the cell membrane, blocking the propagation of lipid peroxidation. Normal vitamin E values (7-8 mg/ml): standardized relative to total cholesterol level (VitE/cholesterol ratio).
Ubiquinone or CoQ10 and in its reduced form ubiquinol or CoQ10H2 (inhibitor of lipid peroxidation). The study of the CoQ10H2/CoQ10 ratio is necessary in order to correctly evaluate the importance of CoQ10 in the protection against AOS attack (detection of patients at risk of cardiovascular problems).
The ratio of glutathione to glutathione reduced by iron (GSH/GSSG) made it possible to obtain a more precise idea of the degree of the oxidative stress. Glutathione peroxidase (GSH) eliminates peroxidized lipids. The assay of erythrocyte glutathione is significant only when the selenium content is less than 60 μg/L.
Homocysteine is an important marker for atherosclerosis. The oxidation of homocysteine has a direct cytotoxic effect on endothelial cells partly linked to the formation of free radicals.

Recent prospective studies have shown that homocysteine levels above 6.3 μmol/l result in a 35% increase in myocardium infarction (normal values: 5 and 15 μmol/l). Correspondence: 5 μmol/l of homocysteine corresponds to an increase of 20 mg/di of total plasma cholesterol, i.e. an increase in cardiovascular risks.

Oxidized homocysteine generates AOSs which, in turn, will oxidize LDLs, particularly in the presence of metals such as iron or copper.

The oxidation of vitamin C to dehydroascorbic acid (intermediate radical: ascorbyl) plays a fundamental role in the regeneration of oxidized vitamin E. A vitamin C level of less than 4 mg/ml reflects an increased risk of coronary artery disease.
The assaying of oxidized lipoproteins (LDLs) fluctuates greatly, thereby requiring the plasma to be stored at −20° C. until the moment of centrifugation.

### 2/DNA Damage:

The assaying of 8-hydroxyguanosine (8-OHG) and the assaying of thiol proteins make it possible to evaluate the DNA damage and to evaluate the level of immune defenses of the organism.
The assaying of 8-hydroxyguanosine (8-OHG) in the urine was not given in this study.
The thiol (—SH) groups react with the activated oxygen species. Albumin has thiol groups.
Oxidized thioredoxins are reduced by thioredoxin reductase (TRxR) (enzyme which has a selenocysteine group in its active site). Thioredoxin-1 is overexpressed in many human tumors, and leads to a decrease in apoptosis. TRxR is involved in the degradation of lipid peroxides and hydrogen peroxide and in the regeneration of the ascorbyl radical to ascorbic acid. It is a selenocysteine flavoprotein.

### 3/Oxidized Proteins/Nitration: Protein Carbonyl Content (PCC):

The demonstration of carbonyl groups in oxidized proteins is the most widely used technique for evaluating oxidized proteins. However, the appearance of these groups tends to reflect an overall modification in the protein and is no doubt not as specifically representative of the presence of an oxidative stress as the detection of hydroxylated Tyr.

### 4/Glucose:

Through auto-oxidation, glucose produces large amounts of ROSs and of glyoxal. The latter binds to the amine group of proteins, resulting in the appearance of carboxylmethyllysine residues. These protein residues will bind copper, and set in motion a lipid peroxidation process, thereby leading to an increase in glyoxal production.

Glucose itself can combine with hemoglobin to give glycosylated hemoglobin (HbA1C). An increase in this marker is found in patients suffering from diabetes.

### 5/Antioxidants:

Dismutase (SOD), Oxygen Radical Absorbance Antioxidant Capacity (ORAC), Trolox equivalent antioxidant capacity (TEAC).

### 6 Zinc:

Zinc is a factor which is a catalyst of heat shock protein functions. A zinc deficiency (<5 mg/l) will block HSP activity. An overload can, conversely, mean exposure to tissue toxicity. It was necessary to correct the deficiencies in the volunteers in order to be able to interpret the results from ingestion of the various compositions.

Zinc protects thiol groups against iron-catalyzed oxidation by binding to the SH functions with an affinity greater than that of iron. It is known that zinc stabilizes copper-zinc superoxide dismutase, facilitating its action.

### 7/Sirtuin Activities:

Biochemical tests for sirt-1 activation, by assaying the activity of histone deacetylases (HDACs): for an identical dosage in each of the two groups, with a different absorption of the oral intakes according to the nycthemeron.
For group G1: 0.002% consumed during the day, without distinction of the time;
For group G2: at an identical concentration, but taken only in the evening.

### CONCLUSIONS

No adverse effects were pointed out that led to
the interruption of the study in human beings after oral administration, and
the intake of a single dose in the subsequent weeks.

Data indicate that adverse effects such as nausea, acid reflux or mild gastrointestinal disorders can occasionally occur, which respond well to symptomatic treatments, without necessarily causing the experiment to be stopped. No evidence of serious drug interactions was observed.

The volunteers of groups G1 and G2 showed no difficulty during ingestion, which was done mainly during food intake.

The intakes of these distinct compositions, belonging to groups G1-G2, showed several differences:

### Results Regarding Clinical Signs

The volunteers of group 2 observed a change improving their well-being, regarding the three criteria retained (sleep, asthenia, and decrease in abdominal panicle) around the 9^{th} day (+/−2 days).

In group 1, the reactions were more rapid, on the 8^{th} day (+/−1 day), but the feeling of well-being was given a mark of 4, whereas, on average, it was 6 in group 2.

The fat wasting, calculated in centimeters, showed a standard deviation of −2.3 cm between group 1 and group 2, in favor of the second group.

### Biological Test Results

The stimulation of sirtuins in group G1 objectivized an increase in HDAC levels:

After 30 days of treatment, an increase in HDAC level was observed that was
62.3%, group G1;
58.9%, group G2.

After 30 days of treatment and 15 days of interruption of the consumption of the compositions, in the two groups, a decrease in HDAC levels was observed that was:
19.3% in group G1;
39% of the basal level in group G2.

The decrease in sirtuin activity is greater in group 1 than in group 2.

For identical doses of active ingredients during the course of the study, a persistence of the beneficial results was observed in group 2, thanks to the observance of the physiological cycle of the cell. The results are summarized in the table below.

| | |
|---|---|
| Group 1 | Group 2 |

Average serum level found for each marker

| | D 0 | D 30 | D 0 | D 30 |
|---|---|---|---|---|
| Glucose | 1.05 +/− 0.12 | 0.95 +/− 0.1 | 0.98 +/− 0.13 | 0.90 +/− 0.10 |
| N: 0.8-1.2 g/l | | | | |
| Vitamin C | 7.38 +/− 5.45 | 9.44 +/− 3.5 | 7.84 +/− 4.77 | 12.56 +/− 3.78 |
| N; 6.5-16.5 μg/ml | | | | |
| Vitamin E | 9.4 +/− 1.82 | 10.14 +/− 2.63 | 9.22 +/− 2.71 | 11.33 +/− 3.05 |
| N: 8-15 μg/ml | | | | |
| Selenium | 95.4 +/− 5.23 | 100.3 +/− 8.6 | 96.23 +/− 4.2 | 113.3 +/− 8.71 |
| N: 94-130 μg/ml | | | | |
| Zinc | 4.6 +/− 0.5 | 5.8 +/− 0.4 | 4.4 +/− 0.5 | 5.5 +/− 0.6 |
| N: 4.5-6 mg/l | | | | |
| Ferritin | 95 +/− 15.3 | 79.4 +/− 5.6 | 78.9 +/− 26.7 | 59.2 +/− 5.7 |
| N: 20-200 ng/ml | | | | |
| CRP | <5 | <5 | <5 | <5 |
| N: <5 mg/l | | | | |
| Albumin | 36.2 +/− 3.6 | 38.2 +/− 0.4 | 35.3 +/− 4.1 | 37.4 +/− 3.8 |
| N: 38-48 g/l | | | | |
| GPX | 67 +/− 10.2 | 76.5 +/− 23.7 | 58.5 +/− 12.05 | 88 +/− 12.07 |
| N: 30-55 UIg/g Hb | | | | |
| SOD | 637 +/− 54 | 793 +/− 77 | 623 +/− 35 | 838 /+− 285 |
| N: 785-1570 UI/g | | | | |
| Hb | | | | |
| Homocysteine | 6.8 +/− 2.3 | 5.7 +/− 0.8 | 7.1 +/− 0.3 | 4.8 +/− 0.3 |
| N: 5-15 μmol/L | | | | |
| Co Enzyme Q 10 | 0.75 +/− 0.23 | 0.82 +/− 0.25 | 0.76 +/− 0.17 | 0.98 +/− 0.22 |
| N: 0.4-1.2 μg/mL | | | | |

In the comparative table, the level of some inflammation markers was identical, with a relatively insignificant deviation (P<0.005), whereas, for other markers:

Either a significant decrease was recorded: ferritin, homocysteine, or an increase was recorded: SOD, CoQ10, GPX, selenium and vitamin C.

There is an analogy between cell epigenetics, including the circadian clock, and the absorption of agonist or antagonist synchronizers which act on cell metabolism. The agonist-antagonist resynchronizing compositions are capable of having an effect on the behavior of cells so as to prevent phenomena of aging thereof and of excess weight, and on the psychological state of the brain.

Regular oral consumption or preferentially oral consumption as treatments, every 3 to 6 months, allows real preventive management.

Persistence of the beneficial effects for a few days to a few weeks may be observed, or even is expected and beneficial, as has already been noted for the preparations of group 2.

1-21. (canceled)

22. A cosmetic, pharmaceutical, food or veterinary combination comprising a first composition containing at least one sirtuin inhibitor and at least one HSP inhibitor, and a second composition containing at least one sirtuin activator and at least one HSP activator.

23. The combination of claim 22, wherein the first composition contains no sirtuin activator and no HSP activator, and the second composition contains no sirtuin inhibitor and no HSP inhibitor.

24. The combination of claim 22, wherein the two compositions are packaged separately.

25. The combination of claim 22, wherein the combination has an increased efficacy in treatment or prevention of an inflammatory process in a patient compared to treatment with either the first or the second composition alone.

26. The combination of claim 22, wherein the combination has an increased efficacy in treatment or prevention of inflammatory processes in a patient compared to treatment with both i) a composition containing sirtuin inhibitors and no HSP inhibitor, and ii) a composition containing sirtuin activators and no HSP activator.

27. The combination of claim 22, wherein the first composition and/or the second composition comprises at least one vascular activating agent.

28. The combination of claim 27, wherein the vascular activating agent is selected from the group consisting of AHAs (Alpha-hydroxy acids), and isoflavons or a plant extract containing same, such as an extract of cassava or an extract of clover, an extract of St. John's wort, an extract of ginkgo biloba, an extract of Sophora japonica, an extract of Centella asiatica, an extract of ruscus (Ruscus aculeatus), an extract of climbing ivy, an extract of agrimony, an extract of mouse-ear hawkweed (Hieracium pilosella), an extract of sweet clover (Melilotus officinalis), an extract of beech buds, an extract of horse chestnut, an extract of dermochlorella, rosavin or a plant extract containing same, such as an extract of Rhodiola rosea plant, and any combination thereof.

29. The combination of claim 22, wherein the sirtuin inhibitor is selected from the group consisting of tocopheryl nicotinate, niacin or a plant extract containing same, sirtinol, cirsimarin, cirsimaritin, capsaicin or a plant extract containing same, extracts of Micotea debilis, the proteins contained in whole cereals, such as peas, lentils, barley, wheat or rye, and also the aqueous extracts of these cereals, and any combination thereof.

30. The combination of claim 22, wherein the HSP inhibitor is selected from the group consisting of deguelin, quercetin, L-glutamine or a plant extract containing same, myricetin, kaempferol, coumestrol, an extract of Sericea mundulea, an extract of red berries containing myricetin, an extract of onion which is a source of quercetin, an extract of caper which is a source of kaempferol, an extract of soya which is a source of coumestrol, and any combination thereof.

31. The combination of claim 22, wherein the sirtuin activator is selected from the group consisting of:
trans-resveratrol or a resveratrol derivative such as diphenyl resveratrol or dihydroresveratrol,
FOXO 3,
xanthohumol or a plant extract containing same, for example an extract of hops,
isoliquiritigenin or a plant extract containing same, for example an extract of liquorice,
phloridzin or a plant extract containing same, for example an extract of apple,
piceatannol or a plant extract containing same, for example an extract of rhubarb,
fisetin or a plant extract containing same, such as an extract of strawberries, of grape, of apple or of tomato,
any combination thereof.

32. The combination of claim 22, wherein the HSP activator is selected from the group consisting of:
TEX-OE or an extract containing same, such as an extract of Barbary fig epicardium,
verbascoside or a plant extract containing same, such as an extract of vervain or an extract of olive,
an extract of Ficus Opuntia indica fruit,
D-trehalose which can be extracted from a brown algae (laminarin) or from Ganoderma lucidum,
rosavin or a plant extract containing same, such as an extract of Rhodiola rosea,
arginine or an extract containing same, for example an extract of walnut,
selenium, and
any combination thereof.

33. The combination of claim 22, wherein the first and/or the second composition also comprises at least one slimming agent which acts:
by stimulating HSL (hormone-sensitive lipase), and/or
by stimulating beta 1/2 adrenergic receptors, and/or
by inhibiting LPL (lipoprotein lipase), and/or
by inhibiting alpha-2 adrenergic receptors, and/or
by blocking adenosine A2a receptors, and/or
by blocking cell differentiation into adipocytes by blocking the induction of peroxisome proliferator-activated receptors (PPARs gamma).

34. The combination of claim 33, wherein the slimming agent is selected from the group consisting of:
tea epigallocathechin-3-gallates (ECGCs) or a green tea cathechin,
luteolin,
forskolin or a plant extract containing same, such as an extract of Coleus,
alpha-linoleic acid (ALA), and
any combination thereof.

35. The combination of claim 22, wherein the first composition contains, per 100 ml of composition:
at least one sirtuin inhibitor selected in the group consisting of:

| | |
|---|---|
| Tocopheryl nicotinate | from 0.001 mg to 1 g |
| Niacin | from 0.001 mg to 1 g |
| Sirtinol | from 0.001 mg to 1 g |
| Aqueous extracts of whole cereals | from 0.001 mg to 1 g of |
| | dry extract |
| Capsaicin | from 0.001 mg to 1 g |
| Co-enzyme Q10 (ubiquinone) | from 0.001 mg to 1 g |
| and mixtures thereof, | |

at least one HSP inhibitor selected in the group consisting of:

| | |
|---|---|
| Quercetin | from 0.001 mg to 3 g |
| Deguelin | from 0.001 mg to 1 g |
| Rice peptide (L-glutamine) | from 0.001 mg to 1 g |
| and mixtures thereof | |

at least one lipolysis activator selected in the group consisting of:

| | |
|---|---|
| Forskolin | from 0.001 mg to 1 g |
| Green tea cathechin | from 0.001 mg to 1 g |
| Luteolin | from 0.001 mg to 1 g |
| Alpha-linoleic acid | from 0.01 mg to 1 g |
| and mixtures thereof | |

and at least one circulation activator selected in the group consisting of:

| | |
|---|---|
| Genistein | from 0.001 mg to 1 g |
| Ginkgo biloba | from 0.001 mg to 1 g |
| Dermochlorella | from 0.001 mg to 1 g |
| Sweet clover | from 0.001 mg to 1 g |
| Horse chestnut | from 0.001 mg to 1 g |
| Arnica | from 0.001 mg to 1 g |
| Witch hazel water | from 0.001 mg to 1 g |
| and mixtures thereof. | |

36. The combination of claim 22, wherein the second composition contains, per 100 ml of composition:
at least one sirtuin activator selected in the group consisting of:

| | |
|---|---|
| Dihydroresveratrol | 0.01 g |
| Fisetin (dehydrated strawberries) | 0.001 g |
| FOXO 3 | 0.001 mg to 1 g |
| and mixtures thereof, | |

at least one HSP activator selected in the group consisting of:

| | |
|---|---|
| TEX-OE | 0.001 mg to 1 g |
| D-Trehalose | 0.001 mg to 1 g |
| Selenium | 0.001 mg to 1 g |
| Rosavin | 0.001 mg to 1 g |
| Arginine | 0.001 mg to 1 g |
| and mixtures thereof, | |

at least one lipolysis activator selected in the group consisting of:

| | |
|---|---|
| Forskolin | from 0.001 mg to 1 g |
| Green tea cathechin | from 0.001 mg to 1 g |
| Luteolin | from 0.001 mg to 1 g |
| Alpha-linoleic acid | from 0.01 mg to 1 g |
| and mixtures thereof, | |

and at least one circulation activator selected in the group consisting of:

| | |
|---|---|
| St. John's wort | from 0.001 mg to 1 g |
| Ginkgo biloba | from 0.001 mg to 1 g |
| and mixtures thereof. | |

37. A method for treating or preventing an illness or a biological dysfunctionning comprising administering to a mammal in need thereof an effective amount of the combination according to claim 22, thereby alleviating at least one symptom associated with the illness or biological dysfunctionning.

38. The method of claim 37, wherein the illness or the biological dysfunctionning is an inflammatory process selected from the group consisting of fat tissues, fibrous cellulite, adipose cellulite, aqueous cellulite, skin aging, obesity, type 2 diabetes, cardiovascular diseases and cancers.

39. The method of claim 37, wherein the illness or the biological dysfunctionning is desynchronization of the circadian rhythm of the central nervous system.

40. The method of claim 37, wherein the administering follows a chronobiological scale over 24 hours indicating the zones suitable for administration of the first and second compositions, so as to obtain a peak optimization of the effectiveness of the first and second compositions, according to a personalized lifestyle.

41. The method of claim 37, comprising a) a step of administering in the day the first composition containing at least one sirtuin inhibitor and at least one HSP inhibitor, and b) a step of administering in the evening the second composition comprising at least one sirtuin activator and at least one HSP activator.

42. The method of claim 41, wherein the step of administering the first composition occurs in the morning, and the step of administering the second composition occurs in the evening.

43. The method of claim 37, comprising administering the combination orally or topically.

44. The method of claim 37, wherein the combination is in a form selected from the group consisting of a beverage, a food source, capsules, tablets or a powder to be diluted.
